(19) United States
(12) Patent Application Publication
(10) Pub. No.: US 2019/0110685 A1
(43) Pub. Date: Apr. 18, 2019

(54) SYSTEMS AND METHODS FOR GUIDED INTERVENTIONAL PROCEDURES

(71) Applicant: General Electric Company, Schenectady, NY (US)

(72) Inventor: Adam Gregory Pautsch, Waukesha, WI (US)

(21) Appl. No.: 15/783,544

(22) Filed: Oct. 13, 2017

(51) Int. Cl. A61B 5/00; A61B 18/18; A61B 34/20

(52) U.S. Cl. CPC A61B 5/0035; A61B 90/11; A61B 34/20; A61B 18/18

(57) ABSTRACT

A system includes an interventional device, a tracking device, an optical tracking system, at least one processor, and a display unit. The interventional device includes an insertion portion and an exterior portion. The insertion portion is configured for use inside of a patient to perform an interventional procedure. The tracking device is disposed proximate to the exterior portion of the interventional device. The optical tracking system is configured to cooperate with the tracking device to provide tracking imaging corresponding to a location and orientation of the tracking device. At least one processor is configured to correlate the tracking imaging information with an anatomical image of the patient to provide a combined image during a medical task for which at least a portion of the insertion portion of the interventional device is disposed inside of the patient. The display unit is configured to display the combined image.

### BACKGROUND OF THE INVENTION

The subject matter disclosed herein relates generally to systems and methods for guided interventional procedures.

Interventional procedures involve the insertion of a device into a patient to perform a medical task. For example, a needle or probe may be inserted into a patient to perform a biopsy or ablation. The path of the needle or probe may be selected to maintain the needle in a position that avoids impact on blood vessels, nerves, or organs. However, such positioning may be time consuming. For example, a number of computed tomography (CT) scans may be performed at different stages during the insertion of the needle or probe as part of a “guess and check” approach to determining needle position and path. Additionally, as the number of CT scans increases, so does the radiation dose received by the patient.

### BRIEF DESCRIPTION OF THE INVENTION

In one embodiment, a system is provided that includes an interventional device, a tracking device, an optical tracking system, at least one processor, and a display unit. The interventional device includes an insertion portion and an exterior portion. The insertion portion is configured for use inside of a patient to perform an interventional procedure. The tracking device is disposed proximate to the exterior portion of the interventional device. The optical tracking system is configured to cooperate with the tracking device to provide tracking imaging information corresponding to a location and orientation of the tracking device. At least one processor is configured to correlate the tracking imaging information with an anatomical image of the patient to provide a combined image during a medical task for which at least a portion of the insertion portion of the interventional device is disposed inside of the patient. The display unit is configured to display the combined image.

In another embodiment, a method is provided that includes inserting an interventional device into a patient. The interventional device includes an insertion portion and an exterior portion. The insertion portion is configured for use inside of a patient to perform an interventional procedure. The interventional device has associated therewith a tracking device that is disposed proximate to the exterior portion of the interventional device. The method also includes acquiring tracking imaging information with an optical tracking system in cooperation with the tracking device. The optical tracking information corresponds to a location and orientation of the tracking device. Further, the method includes correlating the tracking imaging information with an anatomical image of the patient to provide a combined image while the insertion portion of the interventional device is disposed inside of the patient. Also, the method includes displaying the combined image.

In another embodiment, a tangible and non-transitory computer readable medium is provided that includes one or more computer software modules configured to direct one or more processors to: acquire tracking imaging information with an optical tracking system, where the optical tracking information corresponds to a location and orientation of a tracking device, with the tracking device associated with an interventional device, wherein the interventional device comprises an insertion portion and an exterior portion, with the insertion portion configured for use inside of a patient to perform an interventional procedure; correlate the tracking imaging information with an anatomical image of the patient to provide a combined image while the insertion portion of the interventional device is disposed inside of the patient; and display the combined image.

### BRIEF DESCRIPTION OF THE DRAWINGS

FIG. 1 is a schematic block diagram illustrating a system in accordance with various embodiments.

FIG. 2 illustrates a schematic depiction of an interventional device with an integral tracking device in accordance with various embodiments.

FIG. 3 illustrates a schematic depiction of an interventional device with a removably attachable tracking device in accordance with various embodiments.

FIG. 4 illustrates a fiducial marker formed in accordance with various embodiments.

FIG. 5 provides an example display in accordance with various embodiments.

FIG. 6 illustrates a system including a wearable headset display in accordance with various embodiments.

FIG. 7 illustrates an example display provided by the system of FIG. 6.

FIG. 8 illustrates an example optical tracking system in accordance with various embodiments.

FIG. 9 is a flowchart of a method in accordance with various embodiments.

FIG. 10 is a schematic block diagram of an imaging system in accordance with various embodiments.

### DETAILED DESCRIPTION OF THE INVENTION

The following detailed description of certain embodiments will be better understood when read in conjunction with the appended drawings. To the extent that the figures illustrate diagrams of the functional blocks of various embodiments, the functional blocks are not necessarily indicative of the division between hardware circuitry. For example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should be further understood that the figures illustrate example embodiments of the present disclosure. Variations, such as replacing or modifying one or more functional blocks, are possible to achieve similar results.

As used herein, the terms “system,” “unit,” or “module” may include a hardware and/or software system that operates to perform one or more functions. For example, a module, unit, or system may include a computer processor, controller, or other logic-based device that performs operations based on instructions stored on a tangible and non-transitory computer readable storage medium, such as a computer memory. Alternatively, a module, unit, or system may include a hard-wired device that performs operations based on hard-wired logic of the device. Various modules or units shown in the attached figures may represent the hardware that operates based on software or hardwired instructions, the software that directs hardware to perform the operations, or a combination thereof. It may be noted that wireless devices and wireless data transmission may also be utilized.

“Systems,” “units,” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations. Again, wireless devices and wireless data transmission may also be utilized.

As used herein, an element or step recited in the singular and preceded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Also used herein, the phrase “reconstructing an image” is not intended to exclude embodiments in which data representing an image is generated, but a viewable image is not. As used herein, the term “image” broadly refers to both viewable images and data representing a viewable image. It may be noted that various embodiments generate, or are configured to generate, at least one viewable image.

Various embodiments provide systems and methods for tracking of interventional devices (e.g., biopsy needle, ablation probe) in 2D or 3D space using motion capture and/or virtual reality techniques. For example, ceiling mounted cameras or laser systems may be employed. In some embodiments, a fiducial marker is placed on a patient or patient table, and used to calculate probe or needle position relative to the patient on a continuous, ongoing basis as the probe or needle position changes. The determined position may then be used to display the current or updated needle position as an overlay on a screen or other display along with anatomical information of the patient (e.g., anatomical information acquired during an X-ray, CT, MR, or ultrasound imaging scan). In some embodiments, a practitioner (e.g., surgeon or interventional radiologist) may plan an optimal or preferred probe or needle path before beginning an interventional procedure, with the display providing guidance along the optimal or preferred path during the interventional procedure, as well as displaying when the probe or needle is in the correct position. Accordingly, various embodiments save time and reduce radiation dose (e.g., from X-ray or CT scans) relative to conventional “guess and check” approaches. Further, various embodiments provide for more complex needle or probe insertion paths relative to conventional approaches that are limited to axial or near axial slices due to limitations in patient dose and/or image reconstruction time.

Various embodiments provide improved tracking of devices used in connection with interventional procedures. A technical effect of at least one embodiment includes reduction of time required for interventional procedures. A technical effect of at least one embodiment includes reduction of dosage for imaging in connection with interventional procedures. A technical effect of at least one embodiment includes improved ease and accuracy of directing an interventional device inside a patient.

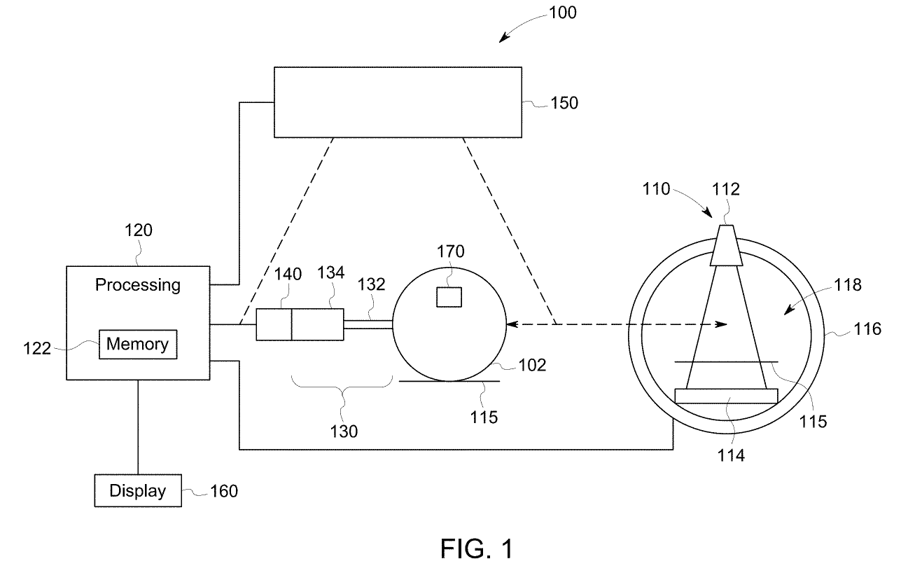

FIG. 1 illustrates a system 100 in accordance with an embodiment. The system 100 may be configured, for example, to perform an interventional procedure, such as an ablation, or, as another example, a biopsy. Generally speaking, a needle or probe is introduced into a patient as part of an interventional procedure to perform a task (e.g., ablation of tissue, collection of a biopsy sample) at a location within the patient. The illustrated embodiment utilizes computed tomography (CT) imaging in connection with guiding an interventional device; however it may be noted that other imaging modalities (e.g., ultrasound) may be employed additionally or alternatively in alternate embodiments.

As seen in FIG. 1, the depicted imaging system 100 includes a CT acquisition unit 110, a processing unit 120, an interventional device 130, a tracking device 140, an optical tracking system 150, and a display unit 160. The components of the system 100 cooperate to guide a practitioner performing an interventional procedure utilizing the interventional device 130 inside patient 102. Generally, the CT acquisition unit 110 is configured to acquire projection data or imaging data (e.g., CT data or CT imaging information) used to generate or reconstruct an anatomical image of the patient 102, the interventional device 130 is configured for use inside the patient 102 to perform an interventional procedure, and the tracking device 140 and optical tracking system 150 are configured to cooperate to provide tracking imaging information corresponding to a location and orientation of the tracking device 140 (and associated interventional device 130). Tracking imaging information may also be referred to as tracking information. The depicted processing unit 120 is configured to correlate the tracking imaging information with an anatomical image of the patient (e.g., a CT image of the patient 102 generated using the CT acquisition unit 110) to provide a combined image (e.g., tracking imaging information or a tracking image overlayed on the anatomical image). The combined image is provided during an interventional procedure using the interventional device 130 (e.g., when at least a portion of the interventional device is disposed inside of the patient 102). It may be noted that the combined image may also be provided when the interventional device 130 is outside of the patient as well. For example, before insertion of the interventional device 130, a combined image showing the position of the interventional device 130 outside of the patient may be provided along with a displayed desired point of insertion and direction or angle of insertion, providing for accurate insertion as well as time savings. The display unit 160 is configured to display the combined image. For example, the display unit 160 may provide the combined image to a practitioner performing an interventional procedure, with the practitioner utilizing the combined image to guide the interventional device 130 to a desired location within the patient.

It may be noted that in various embodiments, the anatomical image may be understood as static, and generated using imaging information acquired before insertion of the interventional device 130 into the patient 102, whereas the tracking imaging information may be understood as dynamic, and updated to provide live needle or probe tracking as the interventional device 130 is advanced toward a target or desired location within the patient 102. It may be noted that various embodiments may include additional components, or may not include all of the components shown in FIG. 1 (for example, various embodiments may provide sub-systems for use with other sub-systems to provide an imaging system). Further, it may be noted that certain aspects of the system 100 shown as separate blocks in FIG. 1 may be incorporated into a single physical entity, and/or aspects shown as a single block in FIG. 1 may be shared or divided among two or more physical entities.

The CT acquisition unit 110 is configured to acquire CT imaging information of the patient 102 (or a portion thereof), and the processing unit 120 is configured to reconstruct or generate an anatomical image using the CT imaging information acquired via the CT acquisition unit 110. It may be noted that, while a CT acquisition unit 110 is depicted in the illustrated embodiment, in other embodiments the component represented by the acquisition unit 110 may utilize one or more other imaging modalities alternatively or additionally. For example, an ultrasound imaging acquisition may be employed. As another example, a multi-modality acquisition unit may be employed. For example, both ultrasound and CT imaging information may be acquired. In some embodiments, ultrasound may be used for a first portion of the patient 102 and CT used for a second portion of the patient 102. The first and second portions may overlap or may not overlap in different embodiments.

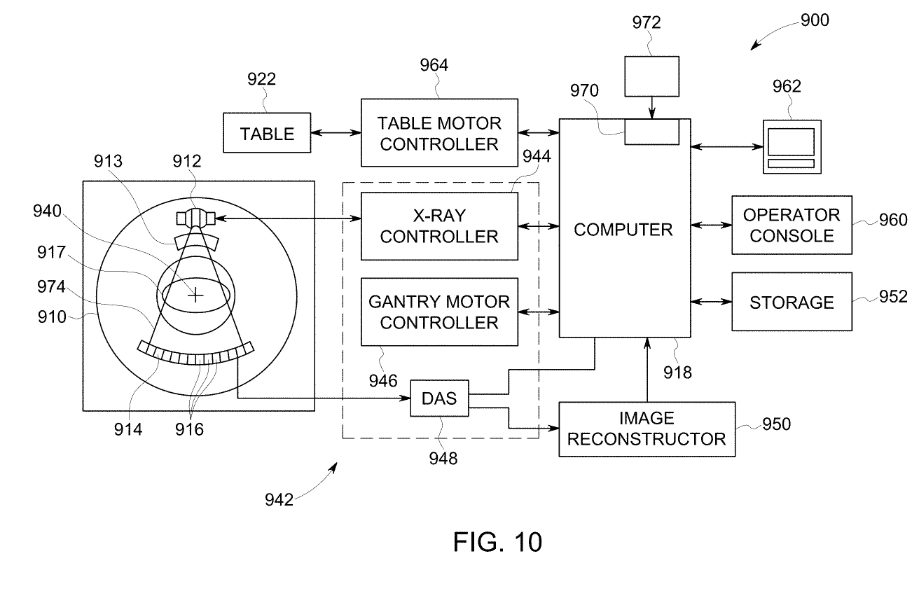

The depicted CT acquisition unit 110 includes an X-ray source 112 and a CT detector 114. (For additional information regarding example CT systems, see FIG. 10 and related discussion herein.) The X-ray source 112 and the CT detector 114 (along with associated components such as bowtie filters, source collimators, detector collimators, or the like (not shown in FIG. 1)) may rotate relative to the object to be imaged. For example, in some embodiments, the X-ray source 112 and the CT detector 114 may rotate about a central axis of a bore 118 of a gantry 116 of the system 100. As another example, the X-ray source 112 and the CT detector 114 may be stationary, while the object spins or rotates about a fixed axis. The system 100 also includes a table 115 that may be advanced into and out of the bore 118 of the CT acquisition unit. The table 115 is configured to support the patient 102.

Generally, X-rays from the X-ray source 112 may be guided to an object to be imaged (e.g., patient 102) through a source collimator and bowtie filter. The object to be imaged in various embodiments is a human patient, or a portion thereof (e.g., portion of patient 102 associated with an interventional procedure to be performed). The source collimator may be configured to allow X-rays within a desired field of view (FOV) to pass through to the object to be imaged while blocking other X-rays. The bowtie filter may be configured to absorb radiation from the X-ray source 112 to control distribution of X-rays passed to the object to be imaged.

X-rays that pass through the object to be imaged are attenuated by the patient 102 and received by the CT detector 114 (which may have a detector collimator associated therewith), which detects the attenuated X-rays and provides imaging information to the processing unit 120. The processing unit 120 may then reconstruct an image of the scanned portion of the patient 102 using the imaging information (or projection information) provided by the CT detector 114.

In the illustrated embodiment, the X-ray source 112 is configured to rotate about an object being imaged. For example, the X-ray source 112 and the CT detector 114 may be positioned about a bore 118 of the gantry 116 and rotated about the patient 102 prior to performance of an interventional procedure. For example, the patient 102 may be positioned on a table 115 outside of the bore 118. Then, the table 115 and patient 102 may be advanced into the bore 118 to acquire CT imaging information. Once the acquisition is complete, the table 115 and patient 102 may be retracted from the bore 118, and the interventional procedure performed with the patient 102 outside of the bore 118. The CT imaging information may be collected as a series of views that together make up a rotation or portion thereof. Each view or projection may have a view duration during which information (e.g., counts) is collected for the particular view. The view duration for a particular view defines a CT information acquisition period for that particular view. For example, each rotation may be made up of about 1000 views or projections, with each view or projection having a duration or length of about 1/1000 of a complete rotation. The X-ray source may be turned on and off to control the acquisition time. For example, to perform an imaging scan of a complete rotation, the X-ray source may be turned on at a particular rotational position of the gantry and turned off when the X-ray source returns to the particular rotational position after a complete rotation. It may be noted that in the illustrated embodiment, the CT acquisition unit 110 is depicted in proximity to other components of the system 100. However, in other embodiments, the anatomical image may be generated from an acquisition unit (e.g., CT and/or ultrasound) that is remotely located away from a location at which the interventional procedure is performed, and may be understood as not forming a part of system 100 in various embodiments.

The interventional device 130 is configured for use inside of the patient 102 for performance of an interventional procedure. As one example, the interventional device 130 may be configured as a needle with a hollow conduit configured for extraction of a sample during a biopsy procedure. As another example, the interventional device 130 may be configured as a probe that delivers energy to a desired location to perform an ablation. The depicted interventional device 130 includes an insertion portion 132 and an exterior portion 134. The insertion portion 132 is generally configured to be used inside of the patient 102. Generally, at least a part of the insertion portion 132 will be disposed inside of the patient 102 during performance of an interventional procedure. The exterior portion 134 is configured for use outside of the patient 102. For example, the exterior portion 134 may include a handle for manipulating the interventional device.

The tracking device 140 is disposed proximate to the exterior portion 134 of the interventional device 130. For example, the tracking device 140 may be part of a handle of the exterior portion 134, or attached to a handle of the exterior portion 134. As another example, the tracking device 140 may be affixed to the exterior portion 134 of the interventional device 130 (e.g., via adhesive, or via one or more fasteners). In other embodiments, the tracking device 140 may be disposed near, but not on the interventional device 130. For example, the tracking device 140 may be affixed to a portion of the insertion portion 132 that is adjacent to or near the exterior portion 134 (e.g., a portion of the insertion portion 132 that is not inserted into the patient 102, and remains outside of the patient 102 during performance of an interventional procedure. Generally, the tracking device 140 is configured as or includes one or more tracking features (e.g., IR sensor chip, reflector) that may be used to determine position and orientation of the tracking device 140 (and accordingly, the interventional device 130 associated with the tracking device 140). It may be noted that the tracking device 140 may be integrally formed with the interventional device 130, or may be removable from the interventional device 130. Further, in embodiments where the tracking device has electronics or circuitry associated therewith, the electronics or circuitry may be mounted to the interventional device 130 or may be located off-board of the interventional device 130 (e.g., coupled to the tracking device 140 via a wire or cable that may be connected and disconnected). Locating electronics or circuitry off-board of the interventional device 130 in various embodiments reduces weight and improves ease of handling the interventional device 130, and also allows the electronics or circuitry to be removed if the interventional device 130 is to be left in the patient 102 for an imaging procedure (e.g., within bore 118 of the CT acquisition unit 110).

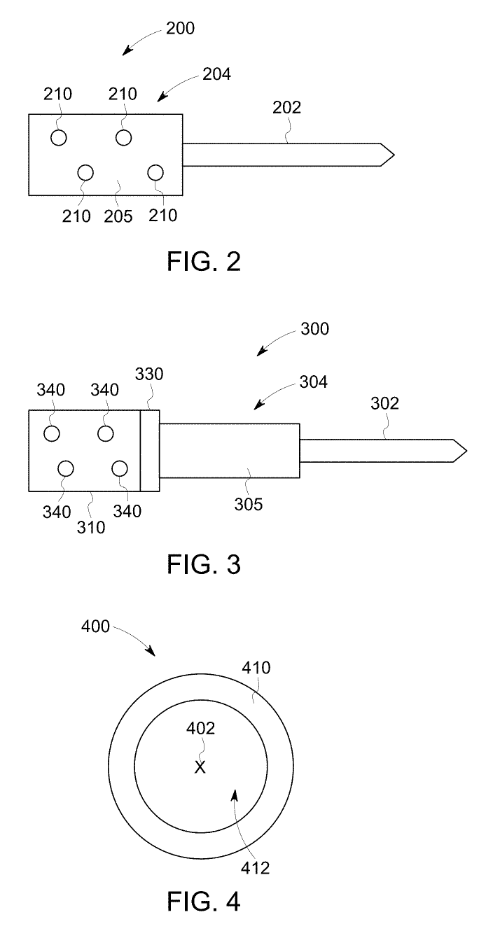

In some embodiments, the tracking device 140 is integrally formed with the interventional device 130. FIG. 2 illustrates a schematic depiction of an interventional device 200 with an integral tracking device 210. As seen in FIG. 2, the interventional device 200 includes an insertion portion 202, and an exterior portion 204. The insertion portion 202, for example, may include a needle. The exterior portion 204 in the illustrated embodiment includes a handle 205. Tracking devices 210 are integrally formed with the handle 205 of the interventional device 200 in the illustrated embodiment. For example, the tracking devices 210 (which may also be referred to as tracking features) may be embedded in or otherwise mounted to the handle 205 during a manufacturing procedure. In some embodiments, the tracking devices 210 are configured as infrared (IR) sensors, such as ASIC sensors, disposed at varying locations about the handle 205, with the sensors configured to detect IR emitted from IR sources positioned about an area in which the interventional device 200 is to be used. In other embodiments, the tracking devices 210 are reflectors configured to reflect a signal (e.g., an IR signal, laser signal) from a source, with the reflected signals detected and used to determine the position of the tracking devices 210 and the position and orientation of the interventional device 200. Use of integral tracking devices 210 provides for consistent placement of tracking devices relative to an interventional device and reduces set-up time for preparing equipment for an interventional procedure.

In some embodiments, the tracking device 140 includes an adaptor that is configured to be removably attached to the interventional device 130, allowing the tracking device 140 and interventional device to be separable. FIG. 3 illustrates a schematic depiction of an interventional device 300 with a removably attachable tracking device 310. As used herein, two objects may be understood as removably attachable with respect to each other when the two objects may be non-destructively separated from each other and re-attached to each other. As seen in FIG. 3, the interventional device 300 includes an insertion portion 302, and an exterior portion 304. The insertion portion 302, for example, may include a needle. The exterior portion 304 in the illustrated embodiment includes a handle 305. The tracking device 310 includes an adaptor 330 and tracking features 340. The tracking features 340 are mounted to or otherwise secured or affixed to the tracking device 310 at known locations, so that the location of the tracking features 340 may be used to determine the position and orientation of the tracking device 310. The depicted adaptor 330 is configured to be removably attached to the handle 305 of the interventional device 300. For example, the depicted adaptor 330 in various embodiments includes one or more features (e.g., slots, tabs, latches, projections, and/or other mating or mounting features) that cooperate with corresponding features on the handle 305 to removably secure the adaptor 330 to the handle 305. In some embodiments, electronics, battery packs, or the like may be removable, with the tracking devices themselves remaining rigidly attached to a probe, with the electronics attached via a wire, thereby minimizing the mass of the probe. Generally, the cooperating features are configured such that the handle 305 and adaptor 330 are in a predetermined spatial relationship or orientation with respect to each other when mated or joined, so that the position of the tracking features 340 disposed on the tracking device 310 may be used to reliably determine the position and orientation of the of interventional device 300 to which the adaptor 330 is mounted. It may be noted that in some embodiments, the tracking features 340 are configured as infrared (IR) sensors, such as ASIC sensors, disposed at varying locations about the tracking device 320, with the sensors configured to detect IR emitted from IR sources (e.g., lighthouses) positioned about an area in which the interventional device 300 is to be used. In other embodiments, the tracking features 340 are reflectors configured to reflect a signal (e.g., an IR signal, laser signal) from a source, with the reflected signals detected and used to determine the position of the tracking features 340 and the position and orientation of the interventional device 300. It may be noted that in various embodiments, IR light sources may be measured by cameras. Further still, in some embodiments, a camera system or other detector may be disposed on the probe and used to track fixed markers disposed external to the probe (e.g., on a wall or ceiling). Accordingly, in some embodiments the tracking device may include sensors or detectors. Yet further still, in some embodiments, accelerometers may be associated with the interventional device 300 and utilized for smoothing or higher speed sampling, and/or be used to stabilize the system in the vent of temporary loss of optical tracking.

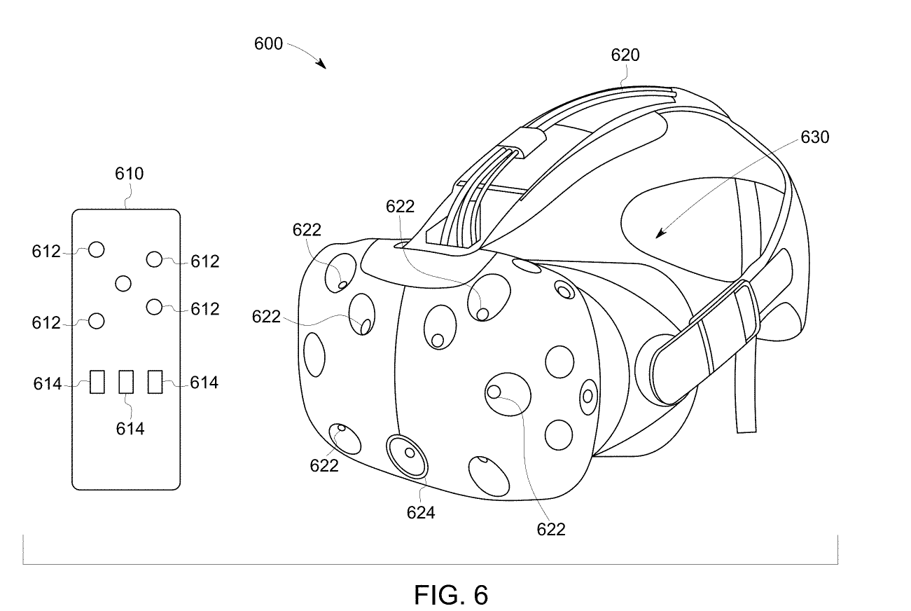

With continued reference to FIG. 1, the optical tracking system 150 is configured to provide tracking imaging information corresponding to a location and orientation (or pose) of the tracking device 140 (e.g., in cooperation with the tracking device 140). Generally, a position and orientation for the tracking device 140 (e.g., determined using tracking features such as reflectors or IR sensors of the tracking device 140) may be used to determine a position or location and orientation (or pose) of interventional device 130 based on a known spatial relationship between the tracking device 140 and the interventional device 130. The optical tracking system 150 in various embodiments includes one or more of a camera, a source of optical signals such as IR signals (e.g., a lighthouse), and/or detectors of optical signals (e.g., IR sensors). Examples of optical tracking systems or components associated with optical tracking systems may be found, for example, in FIGS. 6 and 8 and the related discussion herein.

It may be noted that the particular component (or components) of the system 100 that acquires the tracking imaging information may vary in different embodiments. In some embodiments, the optical tracking system 150 may provide signals that are detected by the tracking device 140. For example, the tracking device 140 in various embodiments uses IR sensors on the tracking device 140 that receive IR signals from the optical tracking system 150, and measures a time difference between a synchronization pulse and signals received at each IR sensor, with the timing information used to determine the position and orientation of the tracking device 140 (and the interventional device 130). In some embodiments, the optical tracking system 150 may provide signals that are reflected off the tracking device 140 (or a portion thereof) using reflectors, with the optical tracking system 150 detecting the reflected signals. The reflected signals are then used to determine the position and orientation of the tracking device 140 (and the interventional device 130). It may further be noted that the position and orientation of the tracking device 140 (and the interventional device 130) in some embodiments, for example, may be determined by the tracking device 140 and/or the optical tracking system 150, with the determined position and orientation information then provided to the processing unit 120. In other embodiments, raw data (such as timing information) may be provided from the optical tracking system 150 and/or the tracking device 140 to processing unit 120, with the position and orientation of the tracking device 140 (and interventional device 130) determined by the processing unit 120. It may be noted that the optical tracking system 150 in various embodiments may include cameras.

Generally, the processing unit 120 in various embodiments correlates the tracking imaging information acquired via the optical tracking system 150 (and tracking device 140) with an anatomical image of the patient 102 to provide a combined image. In various embodiments, the processing unit 120 correlates the tracking imaging information using a fiducial marker 170 that is disposed at a predetermined position relative to the patient 102. In the illustrated embodiment, the fiducial marker 170 is disposed on the patient 102 at a predetermined, measured, or otherwise known location. In other embodiments, the fiducial marker may be placed on a support table or other structure at a known position relative to the patient. Generally, the location of the fiducial marker 170 is used to register the tracking imaging information with the anatomical information. For example, in some embodiments, the fiducial marker 170 may include a feature or tag that is visible in both the anatomical image and the tracking imaging information. In other embodiments, the fiducial marker 170 may be visible in the tracking imaging information (e.g., including a tracking device or feature, or otherwise visible to the optical tracking system 150), and placed at a known position relative to a landmark present in the anatomical image. Multiple fiducial markers may be employed for redundancy and/or improved accuracy of registration of the tracking imaging information to the anatomical image.

In some embodiments, the fiducial marker 170 includes a ring or annular structure. FIG. 4 illustrates a fiducial marker 400 formed in accordance with an embodiment. The fiducial marker 400 is formed as a generally circular ring 410 with an opening 412. The fiducial marker 400 is configured to be adhered or fixed to a patient, for example using an adhesive. In the embodiment illustrated in FIG. 4, the opening 412 of the ring 410 is placed over an anatomical landmark 402 (e.g., navel, nipple) of a patient for convenient, predictable, or reliable placement and positioning of the fiducial marker 400. In various embodiments, a readily identifiable 3D landmark in a CT or Xray image may be employed as the anatomical landmark 402 for accurate, convenient data alignment. In some embodiments, the opening 412 of the ring 410 may be placed over an insertion point for the interventional device 130.

With continued reference to FIG. 1, in various embodiments, the combined image is provided during a medical task for which at least a portion of the insertion portion 134 of the interventional device 130 is disposed inside of the patient 102. For example, in some embodiments, the processing unit 120 provides a combined image in which a representation of the interventional device 130 (using positioning and orientation determined with the optical tracking imaging information) is overlayed on an anatomical representation of the patient 102 (or portion thereof) provided via the anatomical image. Generally, the combined image displays at least a position and orientation of the interventional device 130 relative to the anatomy of the patient 102 provided by the anatomical image. It may be noted that the position and orientation of the interventional device 130 inside of the patient 102 may be determined based on tracking information from the tracking device 140 and optical tracking system 150 outside of the patient 102 using a known spatial relationship between the tracking device 140 and the interventional device 130. The combined image, and particularly the portion of the combined image corresponding to the tracking imaging information, is updated for a live display in various embodiments. For example, the progress of the interventional device 130 into the patient 102 may be tracked and/or guided until the interventional device 130 is in a desired position (e.g., with a tip of a needle at a desired location to collect a sample as part of a biopsy, or with a tip of a probe at a desired location to deliver energy to desired tissue as part of an ablation). In various embodiments, supplemental information or augmented information, such as a desired, planned, or proposed path of the interventional device 130 within the patient 102 may be displayed as well. For example, a practitioner viewing the display can track the progress of the intervention device 130 relative to a desired path, and make appropriate adjustments or otherwise guide the interventional device using the displayed desired path. In various embodiments, the combined display may be understood as a virtual reality (VR) or augmented reality (AR) display. For example, the tracking imaging information may be understood as being displayed against a virtual environment provided by the anatomical image.

Generally speaking, in various embodiments, the anatomical image is acquired, provided, or produced before insertion of the interventional device 130 into the patient 102. For example, the processing unit 120 may reconstruct or generate the anatomical image using imaging information (e.g., CT imaging information, or ultrasound imaging information, among others) acquired with an imaging system or unit. In the illustrated embodiment, the processing unit 120 acquires CT imaging information from the acquisition unit 110 and reconstructs the anatomical image using the CT imaging information. For example, the CT imaging information may be acquired in projection space and transformed into a reconstructed image in image space.

In various embodiments, the processing unit 120 modifies the anatomical image or provides additional or supplemental information. For example, in some embodiments, the anatomical image is modified, or the combined image is modified, so that the combined image includes a marked entry point and/or marked path corresponding to a desired route for the insertion portion. For example, the anatomical image may be displayed to a practitioner via the display unit 160 or other display. Then, based on the internal structures depicted in the anatomical image and the desired end position of the interventional device 130, the practitioner may provide a desired path (and/or entry point) for the interventional device 130 to arrive at a target destination while avoiding internal structures such as blood vessels or organs. Then, during insertion of the interventional device 130, the desired path may displayed along with the anatomical image and tracking imaging information to allow a live comparison of the actual measured or determined position of the interventional device 130 with the desired path, which may be used to guide insertion of the interventional device 130 without requiring CT scans with the interventional device 130 at intermediate steps between insertion and the final, desired end position for performance of the interventional procedure.

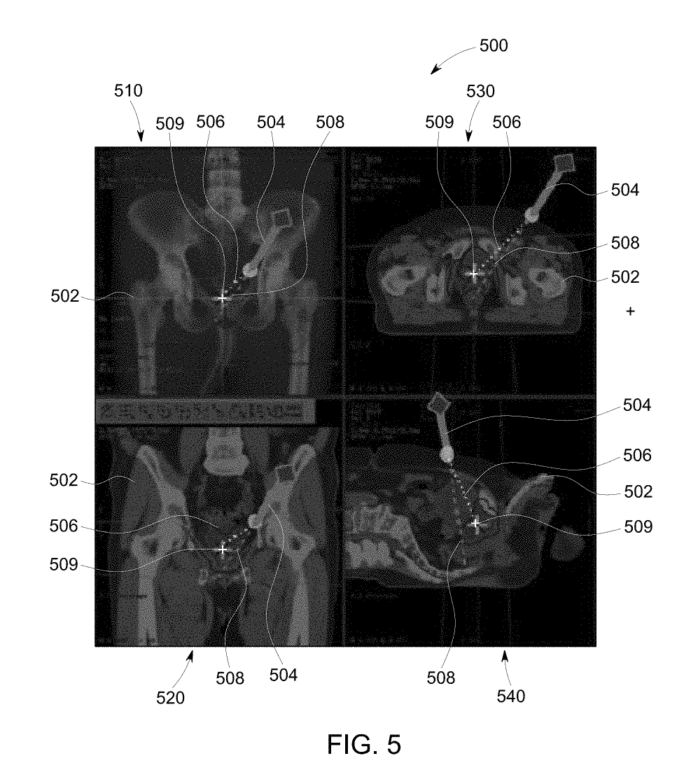

It may be noted that in various embodiments, the desired path may be displayed in 2 and/or 3 dimensions. For example, in some embodiments, the display unit 160 (e.g., under control of the processing unit 120) is configured to show plural views of the marked path. FIG. 5 provides an example display 500 in accordance with an embodiment. The example display 500 includes four views—a first view 510, a second view 520, a third view 530, and a fourth view 540, with each view taken along a different plane or from a different perspective. Each view includes an anatomical portion 502 taken from the anatomical image. Each view also includes an interventional device position portion 504 determined using tracking information and correlated to the anatomical image as discussed herein. Further, each view also includes supplemental information. In the illustrated embodiment, each view includes a desired path 506 corresponding to a predetermined ideal or preferred path of the interventional device, as well as a projected path 508 corresponding to a path the interventional device will follow if it is continued to be inserted deeper into the patient at a current orientation. Accordingly, for example, the practitioner can adjust the orientation of the interventional device until the projected path 508 more closely matches the desired path 506 before further, deeper insertion of the interventional device. Each view also includes a target 509 corresponding to a desired end location of a distal tip of the interventional device.

Returning to FIG. 1, the depicted processing unit 120 is operably coupled to the tracking device 140 and/or optical tracking system 150, the display unit 160, and the CT acquisition unit 110. The processing unit 120, for example, may receive imaging data or projection data from the CT detector 114. Further, the processing unit 120 may receive tracking information from the tracking device 140 and/or optical tracking system 150. Also, the processing unit 120 may provide control signals to one or more aspects of the CT acquisition unit 110, such as the X-ray source 112 and CT detector 114. Further, the processing unit 120 may provide an output (e.g., a combined display as discussed herein) to the display unit 160. In various embodiments, the processing unit 120 includes processing circuitry configured to perform one or more tasks, functions, or steps discussed herein. It may be noted that “processing unit” as used herein is not intended to necessarily be limited to a single processor or computer. For example, the processing unit 120 may include multiple processors and/or computers, which may be integrated in a common housing or unit, or which may be distributed among various units or housings. It may be noted that operations performed by the processing unit 120 (e.g., operations corresponding to process flows or methods discussed herein, or aspects thereof) may be sufficiently complex that the operations may not be performed by a human being within a reasonable time period. For example, the reconstruction of an image, the determination of a position and orientation of the interventional device, and the providing of a combined image may rely on or utilize computations that may not be completed by a person within a reasonable time period.

In the illustrated embodiment, the processing unit includes a memory 122. The memory 122 may include one or more computer readable storage media. For example, the process flows and/or flowcharts discussed herein (or aspects thereof) may represent one or more sets of instructions that are stored in the memory 122 for direction of operations of the processing unit 120 and system 100.

The display unit 160 is configured to provide information (e.g., a combined image as discussed herein) to a user. The display unit 160 may include one or more of a screen, a touchscreen, a printer, or the like. In some embodiments, the display unit 160 may include a wearable headset, for example for use with a VR or AR display.

FIG. 6 illustrates an example system 600 that uses a headset in accordance with an embodiment. The system 600 includes a tracking device 610 and a headset 620. The tracking device 610 is configured to be mounted to or mated with an interventional device (e.g., interventional device 130), and the headset 620 is configured to be worn by a practitioner. The tracking device 610 includes IR sensors 612, and may also include inputs 614 used by a practitioner to input commands (e.g., to the processing unit 120 and/or display unit to control aspects of the display, and/or to provide information). The headset 620 also includes IR sensors 622. The IR sensors 612 and 622 are configured to receive IR signals sent from IR sources (e.g., lighthouses of an optical tracking system), with the received signals used to determine location and orientation of the tracking device 610 and headset 620. In the interior 630 of the headset is a screen (not shown in FIG. 6) providing a display to a user wearing the headset 620. The headset 620 also includes a camera 624 that provides visual information, for example regarding background objects in an environment in which an interventional device being tracked by the system 600 is being used.

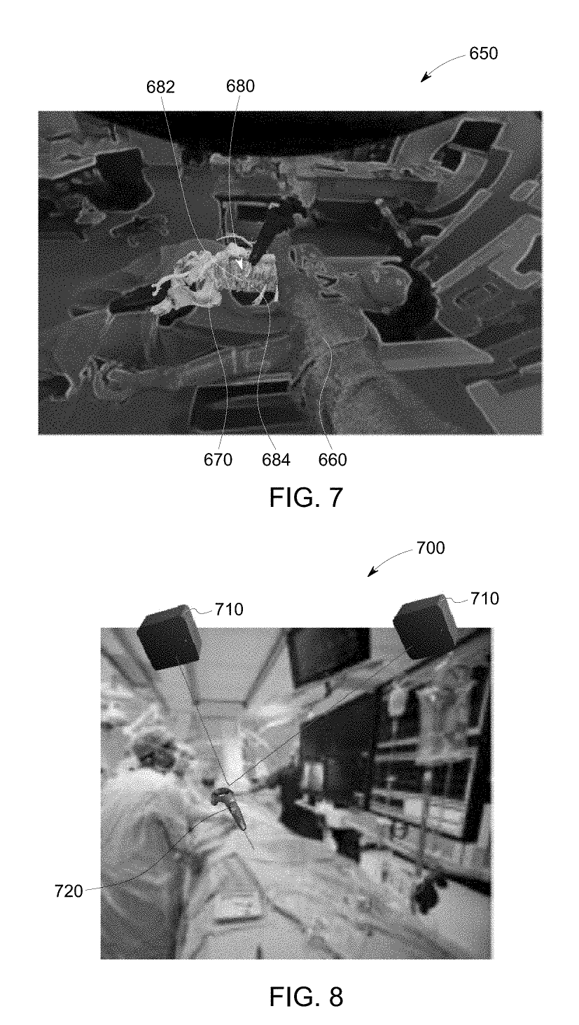

FIG. 7 provides an example view 650 that may be provided by the system 600 to a wearer of the headset 620 during insertion of an interventional device in accordance with various embodiments. As seen in FIG. 7, the view 650 includes environmental information 660, anatomical information 670, and tracking information 680. The environmental information 660, which may be obtained via the camera 624 and/or other cameras, may include for example, portions of the patient beyond the anatomical image, support structures, equipment in the area, or the like. The anatomical information 670, which is provided by the anatomical image previously acquired (e.g., via CT scan) is superimposed on the environmental information. The tracking information 680 includes a determined projected path 682 and a desired path 684 superimposed on the anatomical information 670. The determined projected path 682 is determined in various embodiments utilizing the IR sensors 612 of the tracking device 610, with the path of the interventional device determined based on prior knowledge of the physical or spatial positioning of the interventional device relative to the positions of the IR sensors 612. Accordingly, a practitioner wearing the headset may view the current and projected position of the interventional device relative to the internal anatomy of the patient and/or a predetermined desired path, and guide the interventional device further into the patient accordingly.

As discussed herein, in various embodiments, plural optical signal sources and/or detectors may be utilized. For example, FIG. 8 illustrates a system 700 that includes lighthouses 710 and tracking device 720. In the illustrated embodiment, two lighthouses 710 are shown; however, it may be noted that additional lighthouses may be used to provide redundancy, for example, in case of blockage of signals from one or more of the lighthouses 710. In various embodiments, the lighthouses 710 are mounted at predetermined locations, such as fixed to a ceiling, of an area used for interventional procedures. Generally, the lighthouses 710 provide IR signals detected by the tracking device 720 and used to determine the location and orientation of the tracking device 720. It may be noted that other optical tracking systems, such as those utilizing cameras, may be used additionally or alternatively. Further, the tracking devices in various embodiments may include one or more of sensors (e.g., IR sensors for receiving signals from a lighthouse or base station), reflectors, or optical transmitters (e.g., for transmitting IR or other optical signal detected by a receiver and used to determine position).

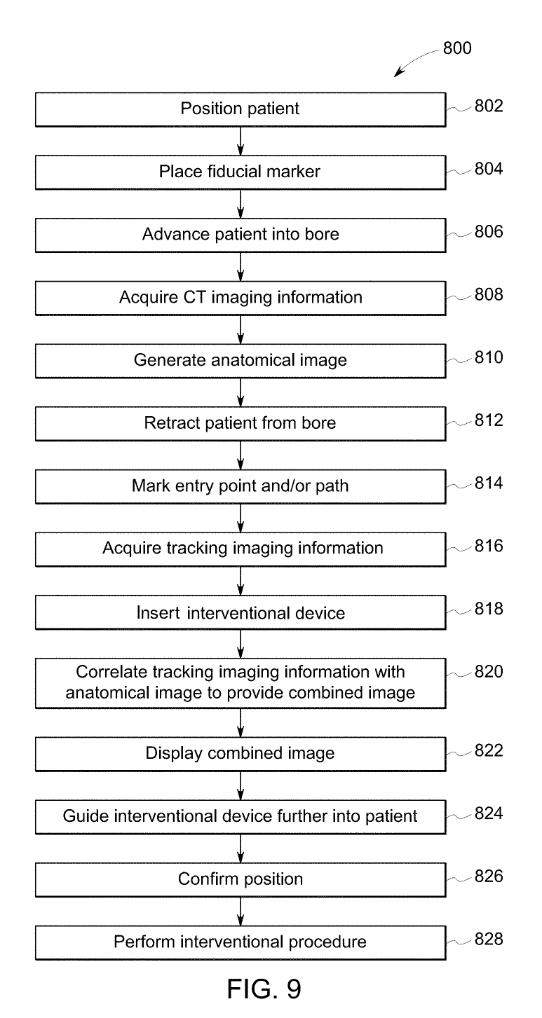

FIG. 9 provides a flowchart of a method 800 for performing an interventional procedure in accordance with various embodiments. The method 800, for example, may employ or be performed by structures or aspects of various embodiments (e.g., systems and/or methods) discussed herein. In various embodiments, certain steps may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 800 may be able to be used as one or more algorithms to direct hardware (e.g., one or more aspects of the processing unit 120) to perform one or more operations described herein.

At 802, a patient is positioned (e.g., on a bed, table, or other support). The patient may also be prepared for an interventional procedure. For example, the patient may be sedated. In the illustrated embodiment, the bed, table, or support is located proximate to and configured for use with an imaging system (e.g., CT imaging system), but it may be noted that in other embodiments imaging information may be acquired at a remote location. At 804, a fiducial marker (e.g., fiducial marker 170) is placed. The fiducial marker in various embodiments may be placed directly on the patient, or in other embodiments may be placed on the table or other structure at a known position relative to the patient (e.g., relative to the portion of the patient being imaged). The fiducial marker is placed for later use in registering imaging information corresponding to internal anatomy of the patient with tracking information corresponding to the position and orientation of an interventional device.

At 806, the patient and support are advanced into the bore of a CT acquisition unit that includes an X-ray source and a detector. It may be noted that in alternate embodiments, a different modality (e.g., ultrasound) may be used additionally or alternatively. At 808, CT imaging information is acquired via the CT acquisition unit. At 810, an anatomical image is generated or reconstructed using the CT imaging information. At 812, the patient and support are retracted out of the bore.

At 814, an entry point and/or marked path are added to the anatomical image. The marked path corresponds to a desired route for an insertion portion of an interventional device within the patient. For example, a practitioner viewing the anatomical image may provide inputs which are used to add the marked path to the anatomical image. It may be noted that this step may be performed on this or a separate system.

At 816, tracking imaging information is acquired with an optical tracking system (e.g., optical tracking system 150). The tracking information corresponds to a location and orientation of the tracking device. With the tracking device at a known spatial relationship with respect to the interventional device, and the tracking devices position and orientation determined using the tracking imaging information acquired in cooperation with the optical tracking system, the position and orientation of the interventional device may be determined. It may be noted that the tracking information may be acquired on an ongoing basis during insertion of the interventional device, with the determined position and orientation of the interventional device updated on an ongoing basis. It may be noted that the tracking imaging information may be initially acquired while the interventional device is outside of the patient. For example, a combined image as discussed herein may be provided before insertion of the interventional device to help guide insertion at a desired point of insertion and at a desired direction or angle of insertion.

At 818, an interventional device (e.g., interventional device 130) is inserted into the patient. For example, an incision may be made at a desired location, and the interventional device inserted into the incision. The interventional device may be configured, for example, for use during a biopsy or ablation. It may be noted that the initial insertion may be quite shallow, with further insertion performed using tracking information as discussed herein. The interventional device includes an insertion portion configured for use inside of a patient (e.g., a needle or probe), and an exterior portion. A tracking device (e.g., tracking device 140) including at least one tracking feature (e.g., IR sensor, reflector) is associated with the interventional device and disposed proximate to the exterior portion of the interventional device. For example, tracking features may be integrally formed with a handle of the interventional device. As another example, an adaptor with one or more tracking features may be removably secured to the interventional device.

At 820, the tracking imaging information is correlated with the anatomical image to provide a combined image. The tracking imaging information and the anatomical image may be correlated using one or more fiducial markers as discussed herein. At 822, the combined image is displayed. The combined image may be displayed on a screen, for example, or, as another example, to a practitioner wearing a headset. The combined image in various embodiments shows the position and orientation of the interventional device overlayed with or relative to the anatomical information. Accordingly, a practitioner viewing the combined image is provided with a visual representation of the interventional device within the interior of the patient, along with surrounding or neighboring anatomical structures. The combined image may also show additional information, such as background or environmental information depicting a surrounding environment, supplemental information including a desired path for the interventional device and/or a projected path of the interventional device based on its current position and orientation, historical information including patient data, or instructional information regarding performance of a given interventional procedure. With the tracking information acquired on an ongoing basis and the position and orientation of the interventional device updated on an ongoing basis, the displayed position of the interventional device may also be updated.

At 824, the interventional device is guided further into the patient using the combined display. As the position and orientation of the interventional device is updated generally continuously or on an ongoing basis, the combined display provides visual feedback for the changing position and orientation of the interventional device as it is further introduced into the patient. For example, the progress of the interventional device may be tracked relative to internal structures, with appropriate adjustments made to avoid the internal structures as the interventional device is advance. As another example, the progress of the interventional device may be tracked relative to a desired path and/or target location, with appropriate adjustments made to match the actual path of the interventional device as measured or determined with the desired path and/or target location.

At 826, with the interventional device advanced to the target location (e.g., as determined using the combined display), the position of the interventional device may be confirmed. For example, the patient, with the interventional device still in place, may be imaged to provide an updated anatomical image. In the illustrated embodiment, the patient may be re-advanced into the bore of the CT imaging acquisition unit, CT imaging information acquired, and a CT image reconstructed showing the position of the interventional device within the patient. If the interventional device is in the desired position, then an interventional procedure may be performed. However, if the interventional device is not in the desired position, the position may be adjusted until it is. It may be noted that in some embodiments the tracking device and/or associated electronics or circuitry may be detached from the interventional device before the patient is advanced into the CT bore, or before the imaging scan to check the position of the interventional device is performed.

At 828, the interventional procedure is performed. With the interventional device in the desired position, it may be used to perform an interventional procedure, such as ablation, or, as another example, to perform a biopsy. After the interventional procedure is complete, the interventional device may be withdrawn from the patient.

Various methods and/or systems (and/or aspects thereof) described herein may be implemented using a medical imaging system. For example, FIG. 10 is a block schematic diagram of an exemplary CT imaging system 900 that may be utilized to implement various embodiments discussed herein. Although the CT imaging system 900 is illustrated as a standalone imaging system, it should be noted that the CT imaging system 900 may form part of a multi-modality imaging system in some embodiments. For example, the multi-modality imaging system may include the CT imaging system 900 and an ultrasound imaging system. It should also be understood that other imaging systems capable of performing the functions described herein are contemplated as being used in different embodiments.

The CT imaging system 900 includes a gantry 910 that has the X-ray source 912 that projects a beam of X-rays toward the detector array 914 on the opposite side of the gantry 910. A source collimator 913 and a bowtie filter are provided proximate the X-ray source 912. In various embodiments, the source collimator 913 may be configured to provide wide collimation as discussed herein. The detector array 914 includes a plurality of detector elements 916 that are arranged in rows and channels that together sense the projected X-rays that pass through a subject 917. The imaging system 900 also includes a computer 918 that receives the projection data from the detector array 914 and processes the projection data to reconstruct an image of the subject 917. The computer 918, for example, may include one or more aspects of the processing unit 120, or be operably coupled to one or more aspects of the processing unit 120. In operation, operator supplied commands and parameters are used by the computer 918 to provide control signals and information to reposition a motorized table 922. More specifically, the motorized table 922 is utilized to move the subject 917 into and out of the gantry 910. Particularly, the table 922 moves at least a portion of the subject 917 through a gantry opening (not shown) that extends through the gantry 910. Further, the table 922 may be used to move the subject 917 vertically within the bore of the gantry 910.

The depicted detector array 914 includes a plurality of detector elements 916. Each detector element 916 produces an electrical signal, or output, that represents the intensity of an impinging X-ray beam and hence allows estimation of the attenuation of the beam as it passes through the subject 917. During a scan to acquire the X-ray projection data, the gantry 910 and the components mounted thereon rotate about a center of rotation 940. FIG. 10 shows only a single row of detector elements 916 (i.e., a detector row). However, the multislice detector array 914 includes a plurality of parallel detector rows of detector elements 916 such that projection data corresponding to a plurality of slices can be acquired simultaneously during a scan.

Rotation of the gantry 910 and the operation of the X-ray source 912 are governed by a control mechanism 942. The control mechanism 942 includes an X-ray controller 944 that provides power and timing signals to the X-ray source 912 and a gantry motor controller 946 that controls the rotational speed and position of the gantry 910. A data acquisition system (DAS) 948 in the control mechanism 942 samples analog data from detector elements 916 and converts the data to digital signals for subsequent processing. An image reconstructor 950 receives the sampled and digitized X-ray data from the DAS 948 and performs high-speed image reconstruction. The reconstructed images are input to the computer 918 that stores the image in a storage device 952. The computer 918 may also receive commands and scanning parameters from an operator via a console 960 that has a keyboard. An associated visual display unit 962 allows the operator to observe the reconstructed image and other data from computer. It may be noted that one or more of the computer 918, controllers, or the like may be incorporated as part of a processing unit such as the processing unit 120 discussed herein.

The operator supplied commands and parameters are used by the computer 918 to provide control signals and information to the DAS 948, the X-ray controller 944 and the gantry motor controller 946. In addition, the computer 918 operates a table motor controller 964 that controls the motorized table 922 to position the subject 917 in the gantry 910. Particularly, the table 922 moves at least a portion of the subject 917 through the gantry opening.

In various embodiments, the computer 918 includes a device 970, for example, a CD-ROM drive, DVD drive, magnetic optical disk (MOD) device, or any other digital device including a network connecting device such as an Ethernet device for reading instructions and/or data from a tangible non-transitory computer-readable medium 972, that excludes signals, such as a CD-ROM, a DVD or another digital source such as a network or the Internet, as well as yet to be developed digital means. In another embodiment, the computer 918 executes instructions stored in firmware (not shown). The computer 918 is programmed to perform functions described herein, and as used herein, the term computer is not limited to just those integrated circuits referred to in the art as computers, but broadly refers to computers, processors, microcontrollers, microcomputers, programmable logic controllers, application specific integrated circuits, and other programmable circuits, and these terms are used interchangeably herein.

In the exemplary embodiment, the X-ray source 912 and the detector array 914 are rotated with the gantry 910 within the imaging plane and around the subject 917 to be imaged such that the angle at which an X-ray beam 974 intersects the subject 917 constantly changes. A group of X-ray attenuation measurements, i.e., projection data, from the detector array 914 at one gantry angle is referred to as a “view” or “projection.” A “scan” of the subject 917 comprises a set of views made at different gantry angles, or view angles, during one or more revolutions of the X-ray source 912 and the detector array 914. In a CT scan, the projection data is processed to reconstruct an image that corresponds to a three-dimensional volume taken of the subject 917. It may be noted that, in some embodiments, an image may be reconstructed using less than a full revolution of data. For example, with a multi-source system, substantially less than a full rotation may be utilized. Thus, in some embodiments, a scan (or slab) corresponding to a 360 degree view may be obtained using less than a complete revolution.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a processing unit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f) unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal language of the claims.

What is claimed is:

1. A system comprising:
an interventional device comprising an insertion portion and an exterior portion, the insertion portion configured for use inside of a patient to perform an interventional procedure;
a tracking device disposed proximate to the exterior portion of the interventional device;
an optical tracking system configured to cooperate with the tracking device to provide tracking imaging information corresponding to a location and orientation of the tracking device;
at least one processor configured to correlate the tracking imaging information with an anatomical image of the patient to provide a combined image during a medical task for which at least a portion of the insertion portion of the interventional device is disposed inside of the patient; and
a display unit configured to display the combined image.

2. The system of claim 1, wherein the an anatomical image comprises a computed tomography (CT) image, the system further comprising:
a CT acquisition unit comprising an X-ray source and at least one CT detector, wherein the CT acquisition unit is configured to acquire CT imaging information and the at least one processor is configured to provide the anatomical image using the CT imaging information.

3. The system of claim 1, wherein the tracking device is integrally formed with the interventional device.

4. The system of claim 1, wherein the tracking device comprises an adaptor configured to be removably attached to the interventional device.

5. The system of claim 1, wherein the at least one processor is configured to correlate the tracking imaging information using at least one fiducial marker disposed at a predetermined position relative to the patient.

6. The system of claim 5, wherein the at least one fiducial marker comprises a ring.

7. The system of claim 1, wherein the combined image comprises a marked entry point and a marked path corresponding to a desired route for the insertion portion.

8. The system of claim 7, wherein the display unit is configured to show plural views of the marked path.

9. The system of claim 1, wherein the display unit comprises a wearable headset.

10. A method comprising:
inserting an interventional device into a patient, wherein the interventional device comprises an insertion portion and an exterior portion, the insertion portion configured for use inside of a patient to perform an interventional procedure, the interventional device having associated therewith a tracking device disposed proximate to the exterior portion of the interventional device;
acquiring tracking imaging information with an optical tracking system in cooperation with the tracking device, the optical tracking information corresponding to a location and orientation of the tracking device;
correlating the tracking imaging information with an anatomical image of the patient to provide a combined image while the insertion portion of the interventional device is disposed inside of the patient; and
displaying the combined image.

11. The method of claim 10, wherein the an anatomical image comprises a computed tomography (CT) image, the method further comprising:
acquiring CT imaging information with a CT acquisition unit comprising an X-ray source and at least one CT detector; and
generating the anatomical image using the CT imaging information.

12. The method of claim 10, wherein the tracking device is integrally formed with the interventional device.

13. The method of claim 10, wherein the tracking device comprises an adaptor configured to be removably attached to the interventional device.

14. The method of claim 10, further comprising disposing at least one fiducial marker at a predetermined position relative to the patient, and using the at least one fiducial marker to correlate the tracking information with the anatomical image.

15. The method of claim 14, wherein the at least one fiducial marker comprises a ring, and wherein disposing the at least one fiducial marker comprises placing the ring around a landmark on the patient.

16. The method of claim 10, further comprising adding a marked entry point and a marked path corresponding to a desired route for the insertion portion to the combined image.

17. The method of claim 16, wherein displaying the combined image comprises displaying plural views of the marked path.

18. The method of claim 10, further comprising:
inserting the interventional device to a desired position; and
performing an interventional procedure after the interventional device is in the desired position.

19. A tangible and non-transitory computer readable medium comprising one or more computer software modules configured to direct one or more processors to:
acquire tracking imaging information with an optical tracking system, the optical tracking information corresponding to a location and orientation of a tracking device, the tracking device associated with an interventional device, wherein the interventional device comprises an insertion portion and an exterior portion, the insertion portion configured for use inside of a patient to perform an interventional procedure;
correlate the tracking imaging information with an anatomical image of the patient to provide a combined image while the insertion portion of the interventional device is disposed inside of the patient; and
display the combined image.

20. The tangible and non-transitory computer readable medium of claim 19, wherein the one or more computer software modules are configured to direct the one or more processors to use at least one fiducial marker to correlate the tracking information with the anatomical image, wherein the at least one fiducial marker is disposed at a predetermined position relative to the patient.
